(19) United States
(12) Patent Application Publication
(10) Pub. No.: US 2026/0240595 A1
(43) Pub. Date: Aug. 20, 2026

(54) APPARATUS FOR PERFORMING MEDICAL PROCEDURES USING RADIOFREQUENCY ENERGY AND RELATED METHODS

(71) Applicant: ElectroWire Corporation, Lexington, SC (US)

(72) Inventors: Robert Leonardi, Lexington, SC (US); Peter J. D'Aquanni, Murrieta, CA (US)

(21) Appl. No.: 19/660,584

(22) Filed: Apr. 28, 2026

Related U.S. Application Data
(63) Continuation of application No. 19/256,282, filed on Jul. 1, 2025.
Application No. 19/256,282 is a continuation-in-part of application No. 18/243,927, filed on Sep. 8, 2023.
(60) Provisional application No. 63/666,745, filed on Jul. 2, 2024.
(60) Provisional application No. 63/405,060, filed on Sep. 9, 2022.

(51) Int. Cl. A61B 18/14; A61B 18/00; A61B 18/12

(52) U.S. Cl. CPC A61B 18/1492; A61B 2018/00357; A61B 2018/00625; A61B 2018/1253

(57) ABSTRACT

An apparatus for performing a minimally invasive medical procedure. The apparatus includes an elongated flexible conductive element, an activator unit, an interventional wire, and an accessory connector. The activator unit is connected to the elongated flexible conductive element and configured to selectively activate the delivery of radiofrequency energy. The interventional wire is electrically connected to the activator unit and configured to deliver radiofrequency energy during the minimally invasive medical procedure. The accessory connector is coupled to the elongated flexible conductive element and configured to connect the elongated flexible conductive element to an electrosurgical unit to deliver continuous, sinusoidal, low-amplitude alternating electrical current from the electrosurgical unit to the elongated flexible conductive element and to the interventional wire. The activator unit is selectively activatable to only deliver the continuous, sinusoidal, low-amplitude alternating electrical current from the electrosurgical unit to the interventional wire.

### CROSS REFERENCE TO RELATED APPLICATIONS

This application is a continuation of U.S. Patent Application No. 19/256,282 filed on July 1, 2025 (pending), which claims the priority of U.S. Provisional Application Serial No. 63/666,745 filed on July 2, 2024. U.S. Patent Application No. 19/256,282 filed on July 1, 2025 is a continuation in part of U.S. Patent Application Serial No. 18/243,927 filed on September 8, 2023 (pending), which claims the priority of U.S. Provisional Patent Application Serial No. 63/405,060, filed on September 9, 2022, the disclosures of which are incorporated herein by reference in their entirety.

### TECHNICAL FIELD

The present disclosure generally relates to the delivery of radiofrequency (RF) energy from a supplier of RF energy to the tip of an interventional wire, via a conductor, to perform medical procedures.

### BACKGROUND

Transcatheter electrosurgery involves the use of energized interventional wires to vaporize tissue. The interventional wires are typically inserted into the cardiovascular system via catheters, which travel to the surgical site through the cardiovascular system. Unlike open electrosurgery, the surgical site in transcatheter electrosurgery is “endovascular,” meaning within the blood-filled space of the cardiovascular system. The electrosurgical energy is supplied by an electrosurgical generator, which may be a dedicated electrosurgical generator designed specifically for a single type of procedure. Alternatively, the electrosurgical generator may be one of many “ubiquitous” electrosurgical generators that are already present in nearly all operating rooms, cardiac catheterization laboratories, and cardiac electrophysiology laboratories. These common electrosurgical generators have industry-standard, three-pin monopolar accessory ports and can generate “cut” (continuous, sinusoidal, low-amplitude), “coagulation” (interrupted, high-amplitude), or various blended waveforms of alternating current in the radiofrequency range of approximately 350kHz to approximately 500 kHz, chosen according to various factors such as the type of procedure being performed.

Existing monopolar electrosurgical accessories (such as electrosurgical “pencils” or “Bovie pencils” for example) are usually connected to conventional, often ubiquitous electrosurgical generators via industry-standard, three-pin monopolar accessory ports. Because existing monopolar electrosurgical accessories are designed for use in open electrosurgery, they have respective activation buttons for cut radiofrequency energy and coagulation radiofrequency energy waveforms. Surgeons are enabled to selectively apply cut and/or coagulation radiofrequency energy waveforms as needed to cut tissue and/or coagulate fluids by using the respective buttons.

Existing monopolar electrosurgical accessories have been adapted for use in transcatheter electrosurgery, enabling cardiologists and cardiovascular surgeons to apply cut radiofrequency energy waveforms to interventional wires as needed to cut and penetrate tissue from within the blood-filled endovascular space. The existing monopolar electrosurgical accessories are connected as usual to common electrosurgical generators via industry-standard, three-pin monopolar accessory ports.

Unlike open electrosurgery, transcatheter electrosurgery uses the application of pure (100%) cut radiofrequency energy waveforms. In transcatheter electrosurgery, pure cut radiofrequency energy waveforms are more effective for cutting tissue than coagulation radiofrequency energy waveforms. Additionally, pure cut radiofrequency energy waveforms are considered to be safer than coagulation radiofrequency energy waveforms in transcatheter electrosurgery. Coagulation radiofrequency energy waveforms, while useful in reducing surgical bleeding during open electrosurgery, are avoided in transcatheter electrosurgery. Coagulation radiofrequency energy waveforms may cause thrombus formation in the blood-filled endovascular space which presents the risk of stroke, myocardial infarction, or other thromboembolic injury. Use of pure cut radiofrequency energy waveforms minimizes these risks. When existing monopolar electrosurgical accessories are adapted for use in transcatheter electrosurgery, the presence of an activation button for coagulation radiofrequency energy waveforms presents the risk that an operator may inadvertently depress the activation button for coagulation radiofrequency energy waveforms, decreasing electrosurgical efficacy and increasing the risk of thrombus formation and thromboembolic injury to the patient.

Accordingly, there is a need for further improvements related to electrosurgical devices .

### SUMMARY

Generally, an apparatus for performing a minimally invasive medical procedure is provided. The apparatus includes an elongated flexible conductive element, an activator unit, an interventional wire, and an accessory connector. The activator unit is connected to the elongated flexible conductive element and configured to selectively activate the delivery of radiofrequency energy. The interventional wire is electrically connected to the activator unit and configured to deliver radiofrequency energy during the minimally invasive medical procedure. The accessory connector is coupled to the elongated flexible conductive element and configured to connect the elongated flexible conductive element to an electrosurgical unit to deliver continuous, sinusoidal, low-amplitude alternating electrical current from the electrosurgical unit to the elongated flexible conductive element and to the interventional wire. The activator unit is selectively activatable to only deliver the continuous, sinusoidal, low-amplitude alternating electrical current from the electrosurgical unit to the interventional wire.

In some embodiments, the accessory connector may be configured to connect to a monopolar accessory receptacle of the electrosurgical unit. The accessory connector may include a three-pin monopolar accessory connector. The accessory connector may be configured to prevent the delivery of interrupted, high-amplitude alternating electrical current to the elongated flexible conductive element. The activator unit may include a switch element configured to selectively activate the delivery of continuous, sinusoidal, low-amplitude alternating electrical current to the interventional wire. The interventional wire may be removably connected to the activator unit. The interventional wire may be configured to serve as a guidewire for delivery of a medical device during the minimally invasive medical procedure when the interventional wire is disconnected from the activator unit.

In alternative embodiments, the interventional wire may include an insulated wire and an uninsulated tip portion. The insulated wire may include a proximal end portion configured for coupling the interventional wire to the activator unit. The uninsulated tip portion may be located at a distal end portion of the insulated wire and configured to deliver radiofrequency energy. The proximal end portion of the interventional wire may include an uninsulated proximal end portion. The elongated flexible conductive element may include a cable. The apparatus may include a coupler configured to electrically couple the activator unit and the interventional wire. The coupler may include a collet configured to electrically couple the activator unit and the interventional wire. The interventional wire may be removably coupled to the collet. The interventional wire may be configured to serve as a guidewire for delivery of a medical device during the minimally invasive medical procedure when the interventional wire is removed from the coupler. The coupler may include a cap configured to couple the interventional wire to the collet. Tightening the cap may compress or tighten the collet on the interventional wire thereby coupling the interventional wire to the collet. Loosening the cap may allow the collet to loosen or expand allowing the interventional wire to be removed from the collet. The apparatus may include a handle and the handle may at least partially contain the coupler. The activator unit may be mounted on the handle.

An alternative apparatus for performing a minimally invasive medical procedure is provided. The apparatus includes an elongated flexible conductive element, an activator unit, a coupler, an interventional wire, and a three-pin accessory connector. The activator unit is connected to the elongated flexible conductive element and configured to selectively activate the delivery of radiofrequency energy. The coupler is electrically connected to the activator unit. The interventional wire is removably connected to the coupler and configured to deliver radiofrequency energy during the minimally invasive medical procedure. The interventional wire is configured to serve as a guidewire for delivery of a medical device during the minimally invasive medical procedure when the interventional wire is removed from the coupler. The three-pin monopolar accessory connector is coupled to the elongated flexible conductive element and configured to connect the elongated flexible conductive element to a monopolar accessory receptacle of an electrosurgical unit to deliver continuous, sinusoidal, low-amplitude alternating electrical current from the electrosurgical unit to the elongated flexible conductive element and to the interventional wire. The activator unit is selectively activatable to only deliver the continuous, sinusoidal, low-amplitude alternating electrical current from the electrosurgical unit to the interventional wire. The accessory connector is configured to prevent the delivery of interrupted, high-amplitude alternating electrical current to the elongated flexible conductive element.

In some embodiments, the activator unit may include a switch element configured to selectively activate the delivery of continuous, sinusoidal, low-amplitude alternating electrical current to the interventional wire. The interventional wire may include an insulated wire and an uninsulated tip portion. The insulated wire may include a proximal end portion configured for coupling the interventional wire to the activator unit. The uninsulated tip portion may be located at a distal end portion of the insulated wire and configured to deliver radiofrequency energy. The proximal end portion of the interventional wire may include an uninsulated proximal end portion. The elongated flexible conductive element may include a cable. The coupler may include a collet configured to electrically couple the activator unit and the interventional wire. The coupler may include a cap configured to couple the interventional wire to the collet. Tightening the cap may compress or tighten the collet on the interventional wire thereby coupling the interventional wire to the collet. Loosening the cap may allow the collet to loosen or expand allowing the interventional wire to be removed from the collet. The apparatus may include a handle and the handle may at least partially contain the coupler. The activator unit may be mounted on the handle.

Generally, a method of performing a minimally invasive medical procedure is provided. The method includes generating radiofrequency energy using an electrosurgical unit, conducting continuous, sinusoidal, low-amplitude alternating electrical current from the electrosurgical unit through an elongated flexible conductive element to an activator unit, selectively activating the activator unit to selectively direct only the continuous, sinusoidal, low-amplitude alternating electrical current to an interventional wire, and using the interventional wire to deliver the continuous, sinusoidal, low-amplitude alternating electrical current to a surgical site.

In some embodiments, the method may include uncoupling the interventional wire from the activator unit and guiding a medical device to the surgical site with the interventional wire. The surgical site may be a heart and the minimally invasive medical procedure may be a transseptal puncture. The activator unit may be selectively activated by activating and deactivating a switch element. The method may include guiding the interventional wire to a surgical site. The method may include guiding the interventional wire through a catheter.

Any of the features and functions described herein may be applied to any of the disclosed embodiments or methods. Additional features and advantages of the inventive aspects disclosed herein will become more apparent upon review of the following detailed description taken together with accompanying drawings of the illustrative and exemplary embodiments.

### BRIEF DESCRIPTION OF THE DRAWINGS

FIG. 1 is a perspective view of an illustrative transcatheter electrosurgical apparatus.

FIG. 2 is an exploded view of a portion of the transcatheter electrosurgical apparatus of FIG. 1.

FIG. 3 is a sectional view of a portion of the transcatheter electrosurgical apparatus of FIG. 1.

FIG. 4 is an illustrative electrical schematic of the transcatheter electrosurgical apparatus of FIG. 1 .

FIG. 5 shows an illustrative electrosurgical generator.

FIG. 6 is a perspective view of a multilayer printed circuit board.

FIG. 7 is a top view of the multilayer printed circuit board of FIG. 6.

FIG. 8 is a top view of a bottom printed circuit board of the multilayer printed circuit board of FIG. 6.

### DETAILED DESCRIPTION

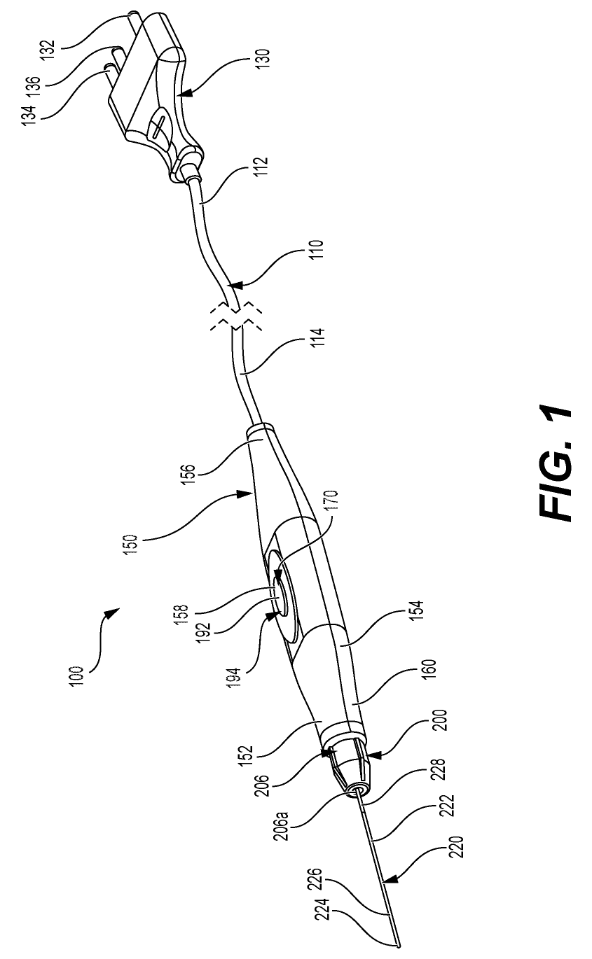

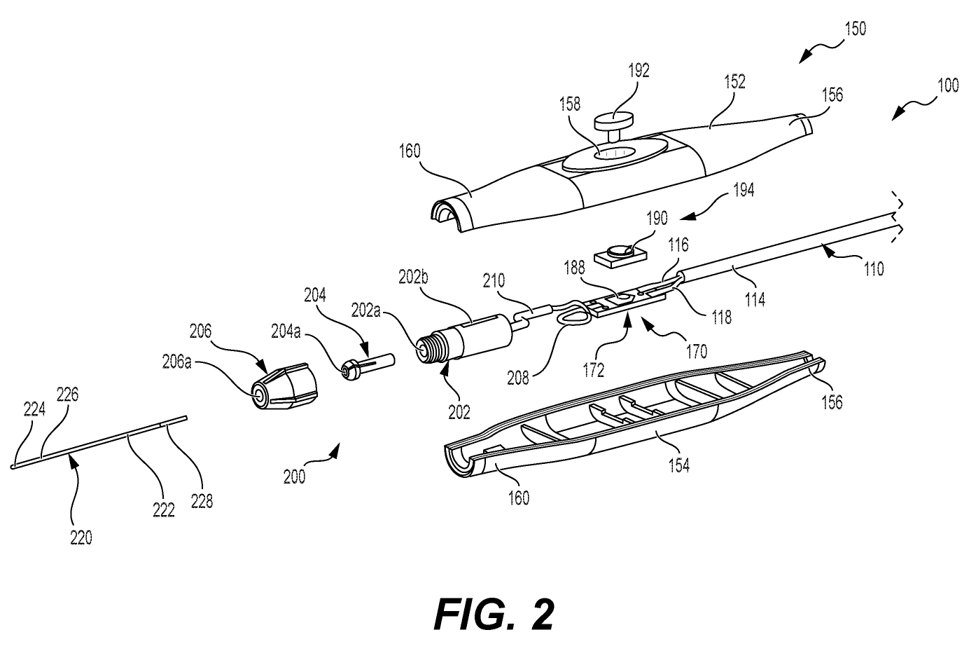

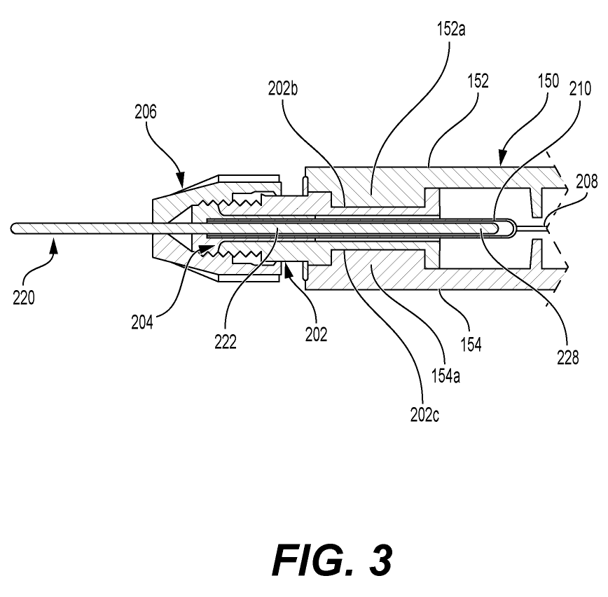

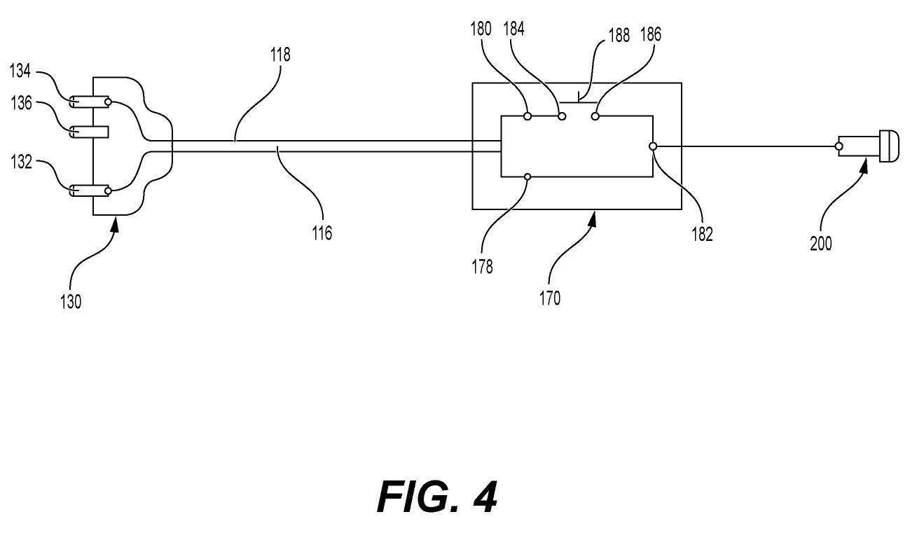

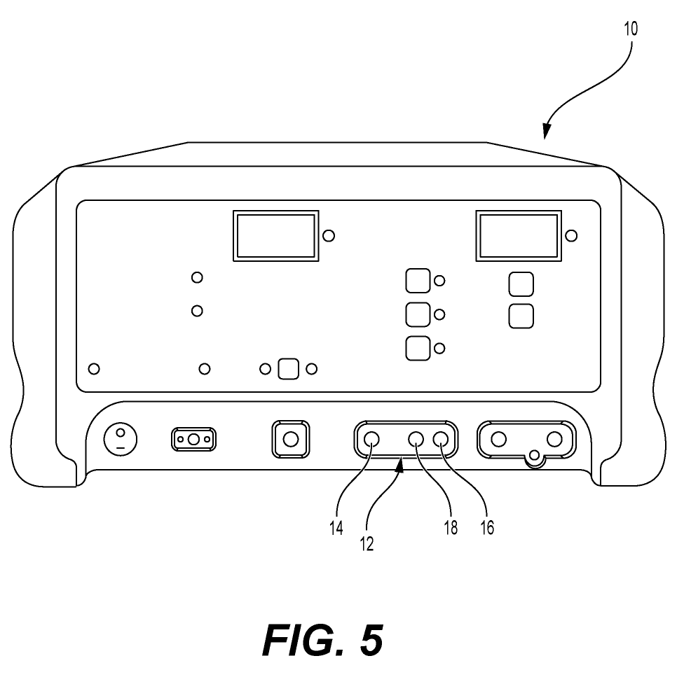

FIG. 1 is a perspective view of an illustrative universally compatible transcatheter electrosurgical apparatus 100 for performing minimally invasive medical procedures. FIG. 2 is an exploded view of a portion of the transcatheter electrosurgical apparatus 100. FIG. 3 is a section view of a portion of the transcatheter electrosurgical apparatus 100. The transcatheter electrosurgical apparatus 100 includes an elongated flexible conductive element 110, an accessory connector 130, a handle 150, an activation unit 170, a coupler 200, and an interventional wire 220. FIG. 4 is an illustrative electrical schematic of the transcatheter electrosurgical apparatus 100. FIG. 5 shows an illustrative electrosurgical generator 10. The electrosurgical generator 10 includes a three-pin monopolar accessory port 12 which has a power socket 14, a cut energy socket 16, and a coagulation energy socket 18. As will be appreciated, the accessory connector 130 is configured to prevent the delivery of interrupted, high-amplitude alternating electrical current (coagulation energy) to the elongated flexible conductive element 110.

Referring to FIGS. 1, 2, and 5, in this illustrative embodiment, the elongated flexible conductive element 110 has a proximal end portion 112 and a distal end portion 114 and is a cable that includes (2) two conductive members or wires 116, 118. The accessory connector 130 is coupled to the proximal end portion 112 of the elongated flexible conductive element 110 and is configured to connect to a three-pin monopolar accessory port 12 of an electrosurgical unit 10, see FIG. 5. The accessory connector 130 includes (3) three pins 132, 134, and 136. Pins 132 and 134 are electrically connected to the wires 116, 118 respectively of the elongated flexible conductive element 110, see FIG. 4. Pin 136 is not electrically connected to the elongated flexible conductive element 110 and may be constructed of conductive and/or non-conductive materials. Pin 136 is located off-center on the accessory connector 130 and ensures that the accessory connector 130 is oriented properly when the accessory connector 130 is connected to a three-pin monopolar accessory port 12 of an electrosurgical unit 10, see FIG. 5. Pin 132 is a powered pin that connects to the power socket 14 and transmits radiofrequency energy from the power socket 14 of the electrosurgical generator 10. Pin 134 is a control pin that connects to the cut energy socket 16 and controls the activation of cut radiofrequency energy.

The accessory connector 130 is configured to deliver cut energy, which is continuous, sinusoidal, low-amplitude alternating electrical current, from the electrosurgical unit 10 to the transcatheter electrosurgical apparatus 100. The accessory connector 130 is also configured to prevent the delivery of coagulation energy, which is interrupted, high-amplitude alternating electrical current, from the electrosurgical unit 10 to the transcatheter electrosurgical apparatus 100. Preventing the delivery of coagulation energy minimizes the risk of thrombus formation in the blood-filled endovascular space thereby minimizing the risk of stroke, myocardial infarction, or other thromboembolic injury.

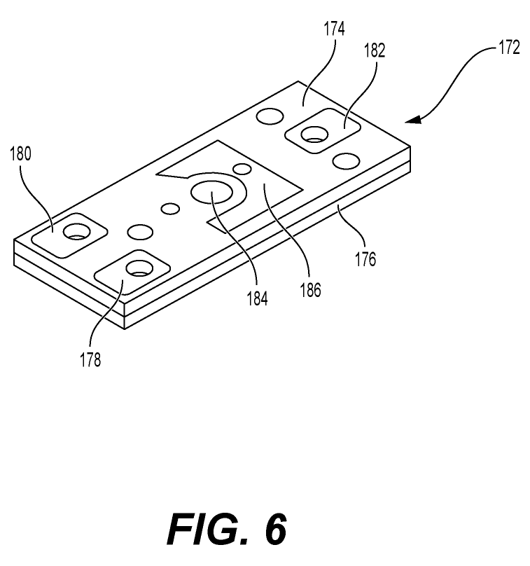

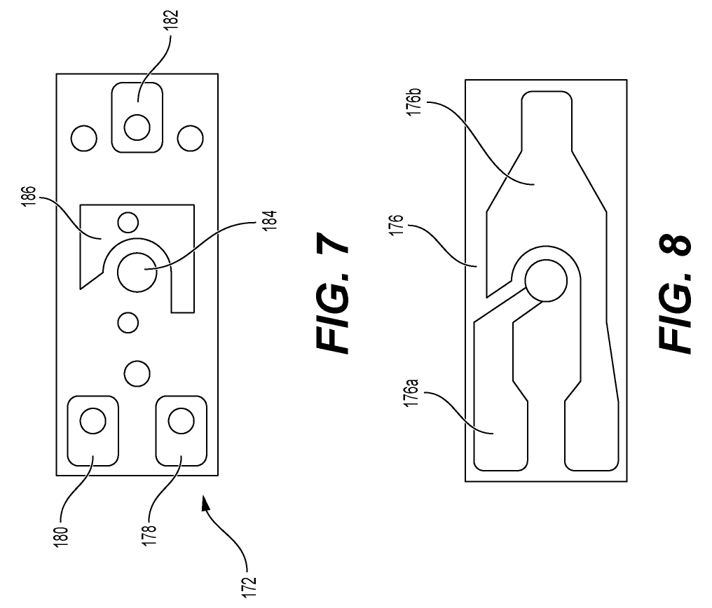

Referring again to FIGS. 1 and 2, in this illustrative embodiment, the handle 150 includes a top handle portion 152 and a bottom handle portion 154 and houses the activation unit 170 and the coupler 200. The top handle portion 152 may be joined to the bottom handle portion 154 by ultrasonic welding, or other means, to prevent fluid ingress into the handle 150 and minimize the risk of unintended application of radiofrequency energy to the operator and/or patient. The activation unit 170 includes a multilayer printed circuit board 172, an electrically conductive snap dome button 188, an electrically insulative silicone pad 190, and a push button 192. FIG. 6 is a perspective view of an illustrative multilayer printed circuit board 172 and FIG. 7 is a top view of the multilayer printed circuit board 172. The illustrative multilayer printed circuit board 172 is constructed from a top printed circuit board 174 and a bottom printed circuit board 176 and includes input connections 178, 180, an output connection 182, and switch contacts 184, 186. FIG. 8 is a top view of the bottom printed circuit board 176 which includes a conductor 176a connecting input connection 180 to switch contact 184. The bottom printed circuit board 176 also includes a conductor 176b connecting input connection 178 to switch contact 186 and output connection 182. The distal end portion 114 of the elongated flexible conductive element 110 passes through the proximal end portion 156 of the handle 150 and is connected to the activation unit 170. The conductor wires 116, 118 of the elongated flexible conductive element 110 are respectively connected to the input connections 178, 180 of the multilayer printed circuit board 172.

The push button 192 passes through a push button hole 158 in the top handle portion 152. Together, the push button 192, silicone pad 190, snap dome button 188, and switch contacts 184, 186 make up a switch element 194. The push button 192 is configured to press the silicone pad 190 which pushes the snap dome button 188 against the switch contacts 184, 186. The snap dome button 188 is a bi-stable metallic structure that creates haptic and audible “snap” feedback when it is actuated by depressing the push button 192. When actuated, the snap dome button 188 creates the electrical bridge across the switch contacts 184, 186, completing the circuit. The electrically insulative silicone pad 190 may reduce the risk of unintended application of radiofrequency energy to the operator and/or patient. The activation unit 170 may include an alternative switch element such as a push button switch or a toggle switch, for example. Note, the button 188 is only used to direct continuous, sinusoidal, low-amplitude alternating electrical current from the electrosurgical unit or generator 10 to the elongated flexible conductive element 110 and to the interventional wire 220. This ensures delivery of cut energy to the interventional wire 220 and there is no coagulation energy activation button which might mistakenly deliver coagulation energy instead.

Referring to FIGS. 1, 2, 3, 4, and 8, in this illustrative embodiment, the coupler 200 is installed at the distal end portion 160 of the handle 150 and includes a coupler housing 202, a collet 204, and a cap 206. The coupler housing 202 and the cap 206 are electrically insulated. The collet 204 is electrically conductive. The coupler housing 202 includes two slots 202b, 202c, located 180 ° apart and configured to be engaged by tabs 152a, 154a of the top and bottom handle portions 152, 154. The tabs 152a, 154a and slots 202b, 202c help maintain the rotational orientation of the coupler housing 202 within the handle 150 during rotation of the cap 206. The collet 204 is connected to the output connection 182 of the multilayer printed circuit board 172 by a conductor wire 208 and a hypotube 210. The distal end of the hypotube 210 is fixed inside the proximal end of the collet 204 by welding or another fixation process. The hypotube 210 is stainless steel and is bent to help maintain the rotational orientation of the collet 204 within the handle 150 during rotation of the cap 206. Alternatively, the conductor wire 208 may be connected to the collet 204 directly or by another conductive metal of another shape or configuration. For example, the collet 204 may be elongated and crimped flat over a segment of its length, serving to secure connection of the collet 204 to the conductor wire 208 and also to help maintain the rotational orientation of the collet 204 within the handle 150 during rotation of the cap 206 by engaging the internal bracing of the top handle portion 152 and the bottom handle portion 154, eliminating the need for a bent hypotube 210. To enable crack-free crimping (flattening) of a segment of the elongated collet 204 while preserving the elasticity of the fingers of the collet 204, the elongated collet 204 may be partially annealed. The collet 204 may be constructed of brass or another electrically conductive and galling resistant material.

The interventional wire 220 is an electrically insulated wire including a proximal end portion 222 configured for coupling the interventional wire 220 to the coupler 200 and an uninsulated tip portion 224 located at a distal end portion 226 of the interventional wire 220 configured to deliver radiofrequency energy during a minimally invasive medical procedure. The interventional wire 220 may be removably connected to the coupler 200. The proximal end portion 222 of the interventional wire 220 is inserted through a hole 206a in the cap 206 and into a hole 204a in the collet 204. The collet 204 is received in a hole 202a into the coupler housing 202. In some embodiments, the proximal end portion 222 of the interventional wire 220 extends through the collet 204 and may be inserted into the coupler housing 202. The proximal end portion 222 of the interventional wire 220 may be inserted through the coupler housing 202 and into the bent hypotube 210 which may serve as a stop to limit the insertion of the interventional wire 220. Alternatively, the flat segment of the elongated collet may serve as a stop to limit the insertion of the interventional wire 220. In this illustrative embodiment, the cap 206 includes internal threads that engage mating threads on the coupler housing 202 and when the cap 206 is tightened, the collet 204 compresses on the interventional wire 220 thereby mechanically connecting the interventional wire 220 to the collet 204 and coupler housing 202 and electrically connecting the interventional wire 220 to the collet 204, the bent hypotube 210, and the activation unit 170. The bent hypotube 210 prevents unwanted rotation of the collet 204 and related twisting of the conductor wire 208 within the handle 150 when the cap 206 is being tightened or loosened. Loosening the cap 206 allows the collet 204 to loosen or expand allowing the interventional wire 220 to be removed from the coupler 200. The coupler 200 may be configured to be compatible with a plurality of interventional wire sizes by varying the size of the coupler housing 202, collet 204, and/or cap 206.

The proximal end portion 222 of the interventional wire 220 may include an uninsulated proximal end portion 228 to ensure a reliable electrical connection to the coupler 200. Another option to using the coupler 200 would be to more directly connect or integrate the interventional wire 220 with the activation unit 170. As desired, the interventional wire may or may not be removably coupled to the activation unit 170. In the illustrated embodiment the interventional wire 220 is configured to serve as a guidewire for delivery of a medical device during a minimally invasive medical procedure when the interventional wire 220 is removed from the coupler 200. In alternative embodiments, the coupler 200 may include a spring mechanism, a push-to-fit mechanism, or similar mechanism for coupling the interventional wire 220 to the coupler 200.

Referring to FIGS. 1, 2, 3, and 5, an illustrative method of preparing the transcatheter electrosurgical apparatus 100 for a medical procedure includes coupling the accessory connector 130 at the proximal end portion 112 of the elongated flexible conductive element 110 to a monopolar accessory receptacle 12 of an electrosurgical unit 10. The electrosurgical unit 10 is configured to generate radiofrequency energy and the activation unit 170 is configured to selectively activate continuous, sinusoidal, low-amplitude alternating electrical current generated by the electrosurgical unit 10. The interventional wire 220 is connected to the coupler 200 by threading and tightening the cap onto the coupler housing 202. When the cap 206 is tightened, the interventional wire 220 is compressed by the collet 204 thereby mechanically and electrically connecting the interventional wire 220 to the coupler 200 and electrically connecting the interventional wire 220 to the activation unit 170. Preparation may include guiding the interventional wire 220 into an introducer sheath and/or a catheter. The electrosurgical unit 10 is energized in preparation for a medical procedure.

Referring to FIGS. 1, 2, and 5, an illustrative method of performing a minimally invasive medical procedure with the transcatheter electrosurgical apparatus 100 includes guiding the interventional wire 220 to a surgical site. The method may include guiding the interventional wire through an introducer sheath and/or catheter. The method includes generating radiofrequency energy using an electrosurgical unit 10. Cut energy, such as in the form of continuous, sinusoidal, low-amplitude alternating electrical current, is conducted from the electrosurgical unit 10 through the elongated flexible conductive element 110 to the activator unit 170. During a minimally invasive medical procedure, when a user depresses the push button 192, the silicone pad 190 presses the snap dome button 188 against the switch contacts 184, 186 of the multilayer printed circuit board 172 thereby selectively activating the switch element 194. Activating the switch element 194 of the activation unit 170 selectively activates the delivery of radiofrequency energy, and specifically, continuous, sinusoidal, low-amplitude alternating electrical current, to the interventional wire 220. The interventional wire 220 delivers the continuous, sinusoidal, low-amplitude alternating electrical current to a surgical site. In some embodiments, the surgical site is a heart and the minimally invasive medical procedure is a transseptal puncture. Loosening the cap 206 allows the collet 204 to loosen or expand allowing the interventional wire 220 to be removed from the coupler 200. Uncoupling the interventional wire 220 from the coupler 200 and activator unit 170 allows the interventional wire 220 to be used as a guidewire. With the interventional wire 220 then detached from the coupler 200, the minimally invasive medical procedure may include guiding a medical device to the surgical site with the interventional wire 220 for performing another part of an overall medical procedure on the patient.

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination within and between the various embodiments. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of the general inventive concept.

What is claimed is:

1. An apparatus for performing a minimally invasive medical procedure comprising:
an elongated flexible conductive element including a distal end and a proximal end configured to be connected to an electrosurgical unit, the elongated flexible conductive element configured to receive and conduct the radiofrequency energy,
a handle,
a coupler mounted on the handle and configured to receive and conduct the radiofrequency energy,
an insulated interventional wire removably coupled to the coupler and configured to deliver the radiofrequency energy during the minimally invasive medical procedure, the interventional wire being configured to serve as a guidewire for delivery of a medical device during the minimally invasive medical procedure when the interventional wire is removed from the coupler, and
an activator unit mounted on the handle and electrically coupled to the distal end of the elongated flexible conductive element and the coupler, the activator unit configured to selectively activate the delivery of the radiofrequency energy from the electrosurgical unit to the interventional wire.

2. The apparatus of claim 1, wherein the interventional wire comprises:
a proximal end portion configured for coupling the interventional wire to the coupler; and
an uninsulated tip portion located at a distal end portion of the interventional wire and configured to deliver radiofrequency energy.

3. The apparatus of claim 2, wherein the proximal end portion of the interventional wire comprises an uninsulated proximal end portion.

4. The apparatus of claim 1, wherein the coupler comprises a collet.

5. The apparatus of claim 4, wherein the coupler further comprises:
a housing at least partially containing the collet; and
a cap coupled to a distal end of the housing;
wherein the interventional wire is coupled to the collet with the cap.

6. The apparatus of claim 5, wherein tightening the cap compresses or tightens the distal end of the collet on the interventional wire thereby coupling the interventional wire to the collet, and
loosening the cap allows the distal end of the collet to loosen or expand allowing the interventional wire to be removed from the collet.

7. The apparatus of claim 1, wherein the activator unit includes a switch element that is configured to selectively supply radiofrequency energy from the electrosurgical unit to the interventional wire when the switch element is activated.

8. The apparatus of claim 1, further comprising an electrosurgical unit connector coupled to the proximal end of the elongated flexible conductive element and configured to connect the elongated flexible conductive element to the electrosurgical unit.

9. The apparatus of claim 8, wherein the electrosurgical unit connector is configured to connect to a monopolar accessory receptacle of the electrosurgical unit.

10. The apparatus of claim 9, wherein the electrosurgical unit connector comprises a three-pin monopolar accessory connector configured to prevent the delivery of interrupted, high-amplitude alternating electrical current to the elongated flexible conductive element.

11. The apparatus of claim 8, wherein the electrosurgical unit connector is configured to couple the elongated flexible conductive element to any one of a plurality of electrosurgical units.

12. An apparatus for performing a minimally invasive medical procedure comprising:
an electrosurgical unit for generating radiofrequency energy; and
an assembly including:
an elongated flexible conductive element including a distal end and a proximal end connected to the electrosurgical unit, the elongated flexible conductive element configured to receive and conduct the radiofrequency energy,
a handle,
a coupler mounted on the handle and configured to receive and conduct the radiofrequency energy,
an insulated interventional wire removably coupled to the coupler and configured to deliver the radiofrequency energy during the minimally invasive medical procedure, the interventional wire being configured to serve as a guidewire for delivery of a medical device during the minimally invasive medical procedure when the interventional wire is removed from the coupler, and
an activator unit mounted on the handle and electrically coupled to the distal end of the elongated flexible conductive element and the coupler, the activator unit configured to selectively activate the delivery of the radiofrequency energy from the electrosurgical unit to the interventional wire.

13. The apparatus of claim 12, wherein the interventional wire comprises:
a proximal end portion configured for coupling the interventional wire to the coupler; and
an uninsulated tip portion located at a distal end portion of the interventional wire and configured to deliver radiofrequency energy.

14. The apparatus of claim 13, wherein the proximal end portion of the interventional wire comprises an uninsulated proximal end portion.

15. The apparatus of claim 12, wherein the coupler comprises a collet.

16. The apparatus of claim 15, wherein the coupler further comprises:
a housing at least partially containing the collet; and
a cap coupled to a distal end of the housing;
wherein the interventional wire is coupled to the collet with the cap.

17. The apparatus of claim 16, wherein tightening the cap compresses or tightens the distal end of the collet on the interventional wire thereby coupling the interventional wire to the collet, and
loosening the cap allows the distal end of the collet to loosen or expand allowing the interventional wire to be removed from the collet.

18. The apparatus of claim 12, wherein the activator unit includes a switch element that is configured to selectively supply radiofrequency energy from the electrosurgical unit to the interventional wire when the switch element is activated.

19. The apparatus of claim 18, further comprising an electrosurgical unit connector coupled to the proximal end of the elongated flexible conductive element and configured to connect the elongated flexible conductive element to the electrosurgical unit.

20. The apparatus of claim 19, wherein the electrosurgical unit connector is configured to connect to a monopolar accessory receptacle of the electrosurgical unit.
